(19) United States
(12) Patent Application Publication
(10) Pub. No.: US 2018/0185674 A1
(43) Pub. Date: Jul. 5, 2018

(54) Patient-Specific Functional Cranioplasty with Integrated Ultrasonic Transmitter Arrangement for Neuromodulation and Opening of the Blood Brain Barrier

(71) Applicants: Ronald Bauer, Teufen (CH); Ralf Schindel, St. Gallen (CH); Erik Dumont, Pessac (FR); Ernst Martin-Fiori, Zurich (CH); Beat Werner, Zurich (CH)

(72) Inventors: Ronald Bauer, Teufen (CH); Ralf Schindel, St. Gallen (CH); Erik Dumont, Pessac (FR); Ernst Martin-Fiori, Zurich (CH); Beat Werner, Zurich (CH)

(21) Appl. No.: 15/740,765

(22) PCT Filed: Jun. 29, 2016
(86) PCT No.: PCT/IB2016/000925

§ 371 Date: Dec. 28, 2017

(30) Foreign Application Priority Data
Jun. 29, 2015 (CH) 00963/2015

(51) Int. Cl. A61N 7/00

(52) U.S. Cl. CPC A61N 7/00; A61B 2090/378; A61N 2007/0078; A61N 2007/0021

(57) ABSTRACT

The invention relates to an ultrasonic transmitter arrangement (1) with a plurality of ultrasonic transmitters (2) arranged on the arrangement whose emittable ultrasonic waves are focused toward a common focus (3). The arrangement (1) is embodied as a cranial bone implant (4) that can be implanted in the cranial bones (5) of a person as a replacement for one or more parts of the cranial bone (5), with the common focus (3) of the entire arrangement being arranged in a defined region of the brain (6) in the implanted state.

The invention relates to an ultrasonic transmitter arrangement and a method for treating a person by means of such an arrangement.

The use of mechanical waves in order to bring about a desired local effect in a local region of a human body through targeted application of mechanical energy is inherently known.

Focused ultrasonic waves can be used to disintegrate kidney stones, for instance. The applied mechanical energy is absorbed in the kidney stones, whereby the kidney stones are reduced to small pieces. Focused ultrasonic waves can also be employed in soft body tissue in order to eliminate diseased or abnormal tissue, such as a tumor or a thrombus that is causing functional disorders in the organism, for example, optionally together with a chemical treatment (tumor ablation, ultrasonically supported thrombolysis).

High-intensity focused ultrasound (HIFU) or low-intensity focused ultrasound (LIFU) can also be used for the treatment of the central nervous system, particularly the brain. The local region in which ultrasonic energy is applied can be monitored using magnetic resonance imaging methods during the ultrasonic intervention in order to enable targeted controlling of the ultrasonic intervention (transcranial magnetic resonance-guided focused ultrasound surgery, tcMRgFUS). Compared to other surgical, chemical, or physical methods of treatment, which oftentimes have negative impacts on patients (risk of hemorrhaging and infection, chemical toxicity, radiation, dose accumulation, etc.), such tcMRgFUS treatment represents a minimally invasive intervention that shows an immediate effect, can be repeated, and can generally be performed on an outpatient basis or with short-term hospitalization.

[symbol]

tcMRgFUS (HIFU or LIFU) has proven to be highly promising as a minimally invasive method for interventions in the brain, being emitted by a plurality of ultrasonic transmitters that are arranged outside of the cranium through the cranial bone and subsequently through other regions of the brain to the region of the brain to be treated (“target region”), where all of the ultrasonic waves converge in a defined focus.

However, the difficulty frequently arises in this method that, upon passing through the cranial bone, the ultrasonic waves that are focused from the outside on the target region in the brain are delayed, deflected, and attenuated to varying degrees depending on the local thickness and density of the bone and on the angle between the direction of propagation (direction of radiation) of the ultrasonic waves (differences in travel time, refraction, and/or absorption by the wall of the cranial bone). For example, at 650 kHz, 90% of the emitted ultrasonic energy is lost on average through absorption in the region of the cranial bone and scalp. The effect of the remaining energy for the intracranial focusing can be reduced further by the following problems:
convergence of the introduced ultrasonic waves at different locations (defocusing, secondary focus formation);
incidence of the introduced ultrasonic energy at different points in time at their respective focus (shifting of travel time); and
occurrence of reflections.

The totality of these phenomena contributes to a significant weakening of the effect of the ultrasonic waves in the target region.

To remedy this situation, the cranial bone can be analyzed by means of computed tomography, which provides insight into the spatial contour of the inner and outer boundary surface of the cranial bone, the internal structure, and the bone density. Based on this information, the spatial orientation (degree of focusing) of the respective ultrasonic transmitter and/or its control signal (pulse phase shift) can be altered in order to compensate for the spatial and temporal faults of the ultrasonic waves in the target region. This compensation of the influence of the cranial bone on the focused ultrasound is quite complicated.

It is the object of the invention to minimize or completely eliminate the difficulties with transcranial magnetic resonance-guided focused ultrasound surgery (tcMRgFUS) described herein.

To achieve this object, the invention proposes a suitable ultrasonic transmitter arrangement with a plurality of ultrasonic transmitters arranged in a distributed manner on the arrangement whose emittable ultrasonic waves are or can be focused toward a common focus, characterized in that the arrangement is embodied as a cranial bone implant that can be implanted into a person's cranial bone as a replacement for a portion thereof, with the common focus of the arrangement lying in a defined region of the brain in the implanted state, or with it being possible for the common focus to be guided there in the implanted state.

The ultrasonic transmitter arrangement according to the invention can be implanted into the patient's cranial bone through a one-time surgical intervention and can serve as a replacement for a portion of his cranial bone. This implant is suitable for LIFU-FUS. LIFU-FUS interventions are used predominantly for a regular to quasi-permanent non-invasive influencing (“neuromodulation”) of a region of the brain that is as small as possible and is responsible for malfunctions of the central nervous system. These interventions are typically reversible; that is, it is possible to temporarily switch off or stimulate a certain local region of the brain that is primarily responsible for a malfunction of the central nervous system. Depending on the neuroanatomical target region and the selected parameters and the duration of the FUS intervention, “temporarily” can mean several minutes, several hours, several days, several weeks, several months, or even longer. By virtue of their being arranged in the implant, the ultrasonic transmitters are always ready at the right place with respect to the defined local region of the brain and can be quickly activated in the event that the LIFU intervention has to be repeated. The invention thus constitutes an alternative to the brain pacemaker (implantation of electrodes into the brain).

The cranial bone implant is preferably embodied as a calotte and, in the implanted state, the ultrasound-emitting regions of the ultrasonic transmitters are arranged on the surface of the calotte facing toward the brain.

This offers the advantage that the ultrasonic waves emitted into the interior of the brain need not pass through any area of the cranial bone, but rather need only propagate through the dura mater and the brain parenchyma to the target region in the brain, so there are no significant differences in the propagation velocity of the ultrasonic waves (phase coherence) and hence practically no deflection of any kind of the ultrasonic waves that are focused toward the target region. The orientations and/or the phases of the control signal of the individual ultrasonic transmitters are therefore much simpler than in the case of an ultrasonic transmitter arrangement outside of the cranial bone.

According to a first embodiment, the calotte is modeled after the removed portion of the cranial bone.

Besides the purely cosmetic aspect, it is thus ensured that the patient experiences as little discomfort as possible from his implant.

According to a second embodiment, the calotte is a spherical shell segment on which the ultrasonic transmitters are arranged so as to be distributed, particularly distributed in a uniform or pseudo-randomized manner, and the common natural focus of the ultrasonic transmitters is the centerpoint of the spherical shell segment.

This enables the use of a spherical ultrasonic transmitter arrangement with a phase-controlled emission characteristic (spherical ultrasound phased array; see antenna diagram of a phase-controlled group antenna), whereby the maximum intensity or the focus of the ultrasonic wave field can be shifted locally.

The structure of the calotte can also be an intermediate form between the first and the second embodiments, so that a compromise is achieved between the advantages of the two designs.

Preferably, the outer edge of the calotte is complementary to the inner edge of the opening in the cranium resulting from the removal of the portion of the cranial bone.

This makes it easier for the surgeon to implant the ultrasonic transmitter arrangement according to the invention in the form of a calotte into the patient's cranial bone through the one-time surgical intervention and/or as a replacement for one or more portions of his cranial bone.

In an especially preferred embodiment, the arrangement is an individualized reconstructed implant that was manufactured on the basis of three-dimensional image data of a person's head, i.e., brain and cranial bone, with the individualized reconstructed implant being preferably manufactured by means of an additive process.

A battery, particularly a rechargeable battery, can be associated with the ultrasonic transmitter arrangement. It can be implanted in the immediate vicinity of the ultrasonic transmitter arrangement. An electric generator, particularly a thermoelectric generator, or a glucose biofuel cell can also be associated with the ultrasonic transmitter arrangement. The electric generator can be implanted in the immediate vicinity of the ultrasonic transmitter arrangement. The electric generator is expediently used to charge the rechargeable battery.

Preferably, a thermoelectric generator is used together with the ultrasonic transmitter arrangement. The thermoelectric generator is preferably implanted into the cranial bone and exploits the temperature gradients between the inner surface and the outer surface of the cranial bone. In order to achieve the object mentioned above, the invention also proposes a method for treating a person who suffers from disorders of the central nervous system, with the method of treatment having the following steps:
identifying a defined region of the person's brain for influencing the underlying pathology;
implanting one or more ultrasonic transmitter arrangements such as those defined in any one of the preceding paragraphs into the person's cranial bone; and
activating of ultrasonic transmitters of the ultrasonic transmitter arrangement in order to feed ultrasonic energy to the defined region of the brain in a targeted manner.

The ultrasonic transmitters can be activated in such a way that the energy of the emitted ultrasonic waves exerts a reversible influence on the defined region of the brain (LIFU). A neuromodulation is brought about in this low-energy mode, or—in conjunction with ultrasound contrast medium, the blood-brain barrier is opened in the target region. Preferably, the blood-brain barrier is opened by means of LIFU-FUS in order to feed very specifically acting pharmaceuticals in the form of macromolecules into the brain and central nervous system. The power can be supplied directly to the transducer elements by means of an implanted pulse generator and supplying electrodes, or it can be supplied transcutaneously by telemetry.

In order to achieve the object mentioned above, the invention additionally proposes a method for preparing a training model for the treatment of a person who suffers from disorders of the central nervous system, with the method having the following steps:
identifying a defined region of the person's brain for influencing the underlying pathology;
acquisition of three-dimensional image data of the person's head, i.e., brain and cranium;
preparation of an individualized reconstructed three-dimensional head model on the basis of three-dimensional image data of the head; and
arrangement of a plurality of ultrasonic transmitters in a cranial bone region of the head model, with the emittable ultrasonic waves being focused as a common focus toward the defined region of the brain of the head model.

This training model can be used to conduct patient- and disease-specific testing by means of HIFU-FUS and LIFU-FUS on the training model.

Preferably, the method for preparing a training model includes an additional step for defining a cutout cranial bone region with ultrasonic transmitters arranged therein as a model for an implant as defined in any one of the preceding paragraphs, as well as a model for a cutting guide for producing an opening in the cranium during a surgical intervention for the implantation of the implant according to the invention.

Additional advantages, features, and possible applications of the invention can be seen from the following non-limitative description of an exemplary embodiment of the ultrasonic transmitter arrangement according to the invention with reference to the drawing, in which

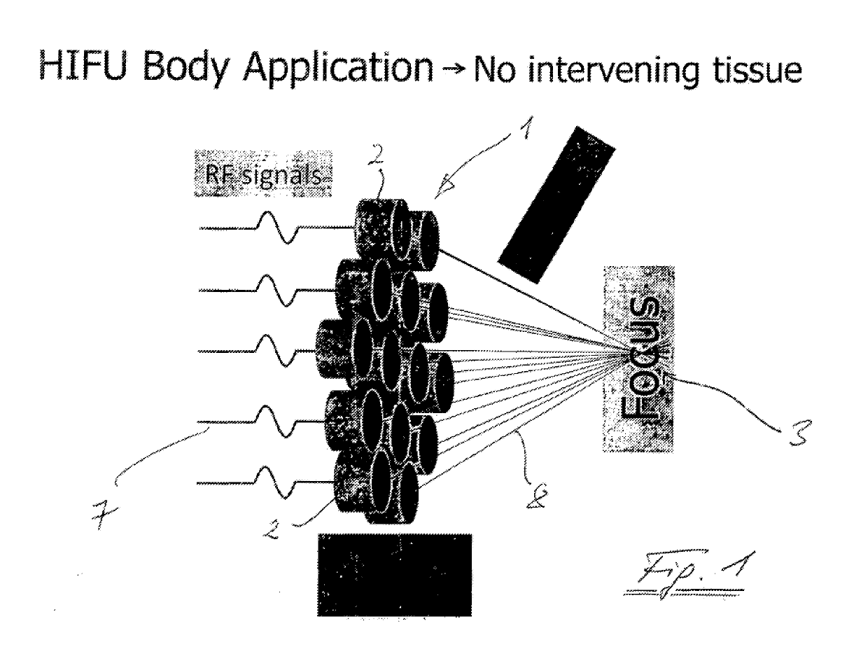

FIG. 1 shows a schematic representation of an ultrasonic transmitter arrangement for focused ultrasound in a first application environment;

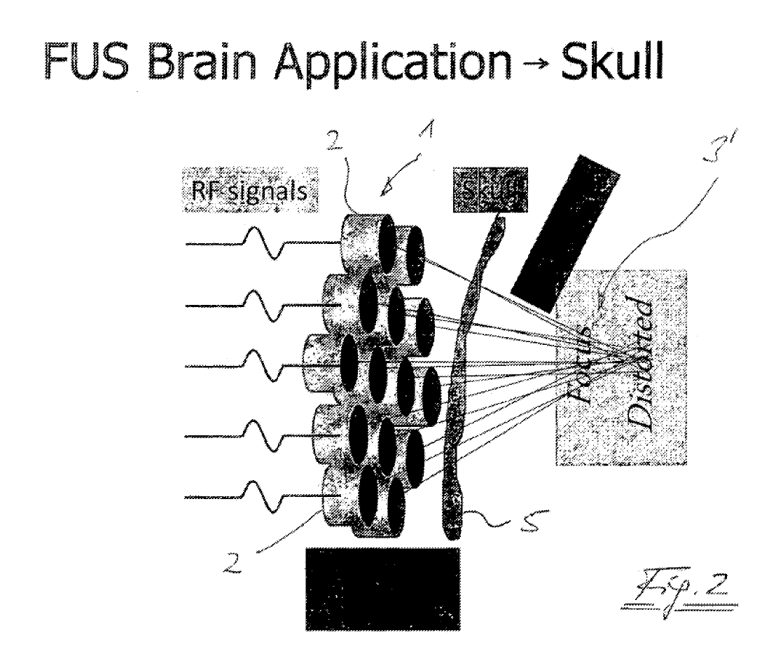

FIG. 2 shows a schematic representation of an ultrasonic transmitter arrangement for focused ultrasound in a second application environment;

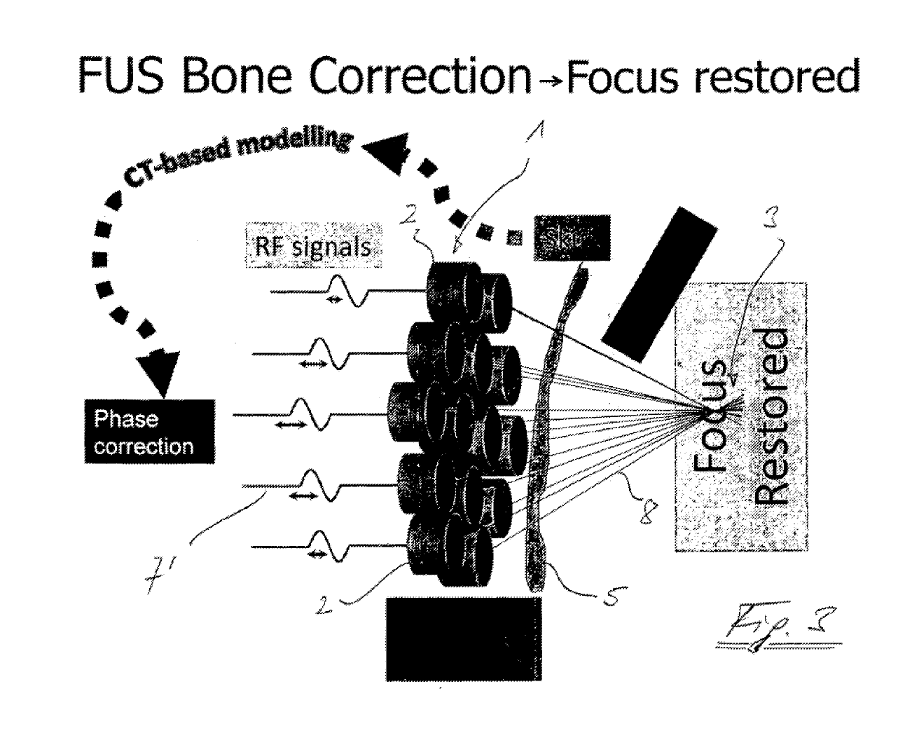

FIG. 3 shows a schematic representation of an ultrasonic transmitter arrangement for focused ultrasound in a third application environment;

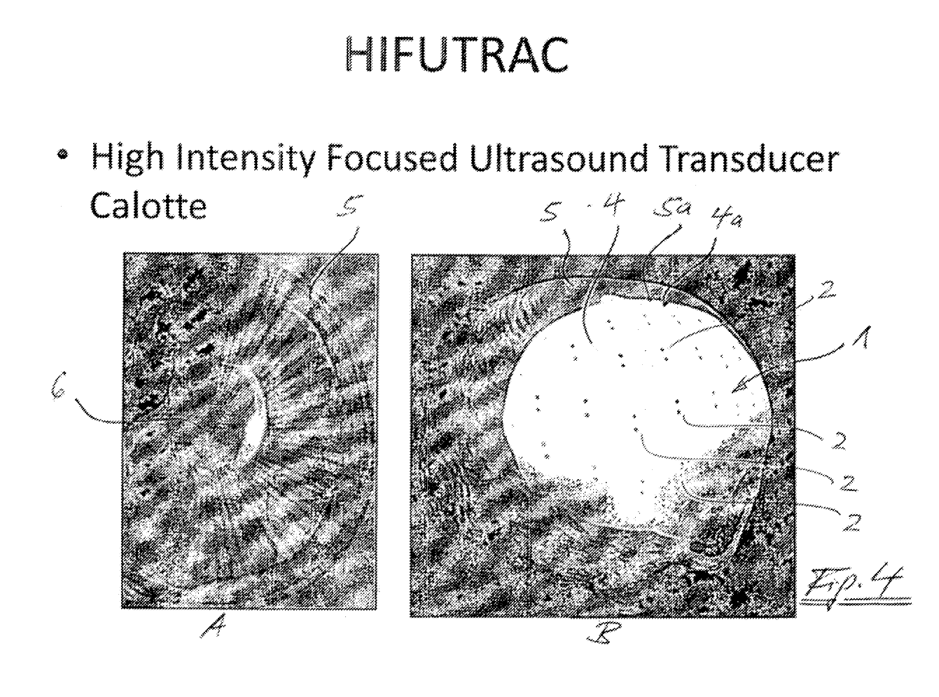

FIG. 4A shows a sectional view of a cranium that is being subjected to a treatment by means of an ultrasonic transmitter arrangement; and

FIG. 4B shows a perspective view of a cranial bone with an implanted ultrasonic transmitter arrangement according to the invention.

FIG. 1 shows a schematic representation of an ultrasonic transmitter arrangement 1 for focused ultrasound in a first application environment. This ultrasonic transmitter arrangement 1 contains a plurality of ultrasonic transmitters 2, each of which is controlled by means of an electrical signal 7 and transmits a focused ultrasonic wave. Instead of the wavefronts, the ultrasonic waves are depicted by means of a beam 8 that is oriented orthogonally to the respective wavefronts. Each of the wavefronts or beams 8 converges in a common focus 3. The intensities of all of the ultrasonic waves add up in the focus 3. The bundled ultrasonic energy of all of the ultrasonic waves is introduced into the tissue at the focus 3 (e.g., bone, connective tissue, muscles, nerves, etc.) or into a foreign body (e.g., kidney stone, blood clot, etc.) and brings about more or less pronounced alterations there through direct mechanical or indirect thermal influence on the tissue or the foreign body.

FIG. 2 shows a schematic representation of an ultrasonic transmitter arrangement 1 for focused ultrasound in a second application environment. The illustration shown in FIG. 2 differs from that of FIG. 1 in that a cranial bone 5 is disposed between the ultrasonic transmitter arrangement 1 and the focus 3. This application environment is used for interventions in the brain by means of focused ultrasound (HIFU or LIFU) as a minimally intensive method, with the ultrasound emitted by the ultrasonic transmitters 2 that are arranged outside of the cranium being emitted through the cranial bone 5 and subsequently through other regions of the brain to the region of the brain to be treated (“target region”) in the focus 3. The aim is for all of the focused ultrasonic waves to converge there (transcranial MRgFUS). The ultrasonic transmitters 2 arranged according to FIG. 1 directs focused ultrasonic waves from the outside to the target region in the brain. Upon passing through the cranial bone 5, the ultrasonic waves are delayed, deflected, and attenuated in their propagation shown in FIG. 1 to varying degrees depending on the local thickness and density of the cranial bone 5 and on the angle between the direction of propagation of the ultrasonic waves and the orientation of the cranial bone 5. The ultrasonic waves irradiated by the ultrasonic transmitters 2 outside of the cranium therefore converge at different locations and, in the case of pulsed ultrasonic waves, even at different points in time at their respective focus. All in all, this results in defocusing 3′ and thus to a massive attenuation of the effect of the ultrasonic waves in the target region in comparison to the effect achieved according to FIG. 1.

FIG. 3 shows a schematic representation of an ultrasonic transmitter arrangement 1 for focused ultrasound in a third application environment. The illustration shown in FIG. 3 differs from that of FIG. 2 in that the individual ultrasonic transmitters 2 of the ultrasonic transmitter arrangement 1 are each controlled by means of a phase-corrected electrical signal 7′ and emit a focused ultrasonic wave with a corrected wavefront and beam direction, which means that, like in FIG. 1, all of the wavefronts and every one of the beams 8 converge again in a common focus 3, and the intensities of all of the ultrasonic waves add up in the focus 3.

FIG. 4A shows an MRI sectional view of a cranium that is being subjected to a treatment by means of an ultrasonic transmitter arrangement 1. One can see the cranial bone 5 surrounding the brain as well as a target region 6 in the brain, which corresponds to the focus 3 of the ultrasonic waves (see FIGS. 1 and 3).

FIG. 4B shows a perspective view of a cranial bone 5 with an implanted ultrasonic transmitter arrangement 1 according to the invention. The ultrasonic transmitter arrangement 1 according to the invention has the shape of a calotte 4 in which a plurality of ultrasonic transmitters 2 are arranged. The outer edge 4a of the calotte 4 is complementary to the inner edge 5a of the opening in the cranium resulting from the removal of the portion of the cranial bone 5. This facilitates the implanting of the calotte 4 in the cranial bone 5 and/or as a replacement for a portion of the cranial bone 5. The calotte 4 is an individualized reconstructed implant that has been manufactured on the basis of three-dimensional image data of the patient's head, i.e., brain and cranial bone, by means of an additive process.

An ultrasonic transmitter arrangement (

1

) comprising:

1. a plurality of ultrasonic transmitters (2) arranged in a distributed manner on the arrangement whose emittable ultrasonic waves are or can be focused toward a common focus (3);
wherein the arrangement (1) is embodied as a cranial bone implant (4) that can be implanted into a person's cranial bone (5) as a replacement for a portion thereof; and
wherein the common focus (1) of the arrangement lying in a defined region (6) of the brain in the implanted state, or with it being possible for the common focus (3) to be guided there in the implanted state;

2. The ultrasonic transmitter arrangement (1) as set forth in claim 1, wherein the cranial bone implant (4) is embodied as a calotte and, in the implanted state, the ultrasound-emitting regions of the ultrasonic transmitters (2) are arranged on the surface of the calotte (4) facing toward the brain.

3. The ultrasonic transmitter arrangement (1) as set forth in claim 2, wherein the calotte (4) is modeled after the removed portion of the cranial bone (5).

4. The ultrasonic transmitter arrangement (1) as set forth in claim 2, wherein the calotte (4) is a spherical shell segment on which the ultrasonic transmitters (2) are arranged so as to be distributed, and the common natural focus of the ultrasonic transmitters (2) is the centerpoint of the spherical shell segment.

5. The ultrasonic transmitter arrangement (1) as set forth in claim 4, wherein the outer edge (4a) of the calotte (4) is complementary to the inner edge (5a) of the opening in the cranium resulting from the removal of the portion of the cranial bone (5).

6. The ultrasonic transmitter arrangement (1) as set forth in claim 1, wherein the arrangement is an individualized reconstructed implant that was manufactured on the basis of three-dimensional image data of a person's head, i.e., brain and cranial bone.

7. The ultrasonic transmitter arrangement as set forth in claim 6, wherein the individualized reconstructed implant was manufactured by means of an additive process.

8. The ultrasonic transmitter arrangement as set forth in claim 1, wherein a battery, particularly a rechargeable battery, is associated therewith.

9. The ultrasonic transmitter arrangement as set forth in claim 1, wherein a generator for electrical energy, particularly a thermoelectric generator or a glucose biofuel cell, is associated therewith.

10. A method for treating a person who suffers from disorders of the central nervous system, the method comprising:
identifying a defined region of the person's brain for influencing the underlying pathology;
implanting an ultrasonic transmitter arrangement comprising of a plurality of ultrasonic transmitters into the person's cranial bone; and
activating one or more of said plurality of ultrasonic transmitters of the ultrasonic transmitter arrangement in order to feed ultrasonic energy to the defined region of the brain in a targeted manner.

11. The method as set forth in claim 10, wherein the ultrasonic transmitters are activated in such a way that the energy of the emitted ultrasonic waves exerts a reversible influence on the defined region of the brain. (LIFU)

12. A method for manufacturing a training model for treating a person who suffers from disorders of the central nervous system, the method comprising
identifying a defined region of the person's brain for influencing the underlying pathology;
acquisition of three-dimensional image data of the person's head, i.e., brain and cranium;
preparation of an individualized reconstructed three-dimensional head model on the basis of three-dimensional image data of the head; and
arrangement of a plurality of ultrasonic transmitters in a cranial bone region of the head model, with the emittable ultrasonic waves being focused as a common focus toward the defined region of the brain of the head model.

13. The method as set forth in claim 10, further comprising defining of a cut-out region of the cranial bone with the ultrasonic transmitters arranged therein as a model for an implant as well as a model for a cutting guide for producing an opening in the cranium during a surgical intervention for the implantation of the implant.

14. The ultrasonic transmitter arrangement (1) as set forth in claim 1, wherein said implant can be applied as a single or multiple element implant.
